(19) United States
(12) Patent Application Publication
(10) Pub. No.: US 2023/0129948 A1
(43) Pub. Date: Apr. 27, 2023

(54) ENHANCED ACNE TREATMENT COMPOSITION

(71) Applicant: BLISTEX INC., Oak Brook, IL (US)

(72) Inventors: Sunanda Neupane, Naperville, IL (US); Anna Pavlovic, Elmwood Park, IL (US); Richard M. Brucks, Chicago, IL (US); Wanda Richard, Plainfield, IL (US); Tommaso Sardone, Naperville, IL (US)

(21) Appl. No.: 17/451,739

(22) Filed: Oct. 21, 2021

(51) Int. Cl. A61K 31/60; A61K 9/00; A61K 47/12; A61K 47/02

(52) U.S. Cl. CPC A61K 31/60; A61K 9/0014; A61K 47/12; A61K 47/02

(57) ABSTRACT

The present invention relates to a composition for topical application to human skin comprising by weight: about 0.01% to about 35% salicylic acid, about 0.01% to about 65% solubilizing, agent selected from one or more alkali and alkaline earth hydroxides, alkali and alkaline earth metal salts of organic acids, wherein the solubilizing agent is present in an amount sufficient to solubilize the salicylic acid in the absence of alcohol, a cosmetically acceptable aqueous medium in which the salicylic acid and solubilizing agent are dissolved.

### FIELD OF THE INVENTION

This invention relates to vehicles for the percutaneous delivery of at least one pharmaceutically active agent to the epidermis. In particular this invention relates to pharmaceutical compositions directed to the treatment of skin diseases, including acne, more particularly those containing salicylic acid.

### BACKGROUND OF THE INVENTION

Pharmaceutical active agents commonly used to treat acne and other skin diseases include but are not limited to the therapeutic substances' salicylic acid, isotretinoin, benzoyl peroxide, resorcinol, non-steroidal anti-inflammatory drugs such as ketoprefen, corticosteroids such as cortisone, antifungals, antibiotics for microbial infections, and anti-psoriatics such as etrinate. It is common to also include other substances such as anesthetics, for example lignocaine where necessary. Treatment of acne is traditionally affected by external, topical application of a pharmaceutical substance. Where this is ineffective, systemic treatments such as by hormone treatment can be utilized but may have undesirable side effects in some patients. Salicylic acid is one well recognized anti-acne active agent which causes a reduction in intercellular cohesion on individual dead skin cells which are the starting point of acne infection. Ideally an anti-acne pharmaceutical should maximize penetration of the active agent through the upper layers of the epidermis and should assist in optimizing the levels of active agent retained in the epidermis without allowing penetration of the active agent into the patient's system.

The challenge in applying a pharmaceutical topically is to achieve percutaneous penetration of the active agent to the site of treatment, in many cases the epidermis. At the same time, it is important that the composition have desirable cosmetic characteristics. Application should be easy, smooth and should result in no irritation, discomfort, or inconvenience. Desirably the composition should not leave a residue on the surface of the skin, oily or otherwise. Active agents can be applied in various vehicles such as liquid preparations, mousses, gels, ointments, lotions, creams and pastes. Such compositions are often very viscous requiring substantial rubbing to achieve penetration of the active agent to the affected skin layer, an act which often results in discomfort and further irritation. Non viscous creams and lotions require quick and dexterous application as they are inclined to flow off the site of treatment before penetration of the active agent is achieved. As solution pharmaceuticals can be difficult to apply because they evaporate due to the heat of the skin surface before penetration to the affected site can be achieved. Many conventional pharmaceutical treatments for acne contain alcohol, usually primarily as a diluent for active ingredients, such as salicylic acid and the like. Notwithstanding, the use of alcohol is non-desirable for a number of reasons, including that it dilates the pores of the skin leading to blackheads and whiteheads, and defeating the purpose of any acne treatment. Alcohol may also cause inflamed skin papules (lesion-like bumps) and cystic acne, and in the long term may cause aging of the skin and possibly even permanent scarring. Alcohol is also known to dry the skin, which can exacerbate irritation in the presence of salicylic acid. It is to be avoided if possible.

In general terms, it is an object of this invention to provide a vehicle for percutaneous delivery of an active agent which is an alternative to those described in the prior art and which provides both high level penetration of the active agent to the site of the treatment, and minimal penetration of the active agent past the skin into general circulation.

It is another object to provide a pharmaceutical composition suited to the treatment of acne which is cosmetically acceptable as well as being pharmaceutically effective.

It is still a further object of this invention to provide such a pharmaceutical composition comprising salicylic acid, and be alcohol free, and which has economic benefits in ease of preparation.

Throughout the specification the term “vehicle” means a composition which has only excipients or components required to carry an active agent, but which itself has no pharmaceutical or therapeutic effect. The term “active agent” means a substance having a pharmaceutical, pharmacological or therapeutic effect in the absence of any excipient. A “pharmaceutical composition” is one having at least one active agent in a vehicle formulated to deliver the active agent to the site of treatment. The term “comprises/comprising” when used in this specification is taken to specify the presence of stated features, integers steps or components but does not preclude the presence or addition of one or more other features, integers, steps, components, or groups thereof.

### SUMMARY OF THE INVENTION

To this end there is provided a non-alcoholic, non-borate containing vehicle for percutaneous delivery of salicylic acid to the epidermis, or acne treatment.

### DETAILED DESCRIPTION OF PREFERRED EMBODIMENTS OF THE INVENTION

The salicylic acid which is the active ingredient of the composition of the invention must be adequately solubilized in the absence of the alcohol. The term “alcohol” as used herein refers to the lower alkanols, in particular, methanol, ethanol, propanol and isopropanol.

Salicylic acid occurs in the form of acicular crystals or crystalline powder with a melting point of 157-159° C. and a strong tendency to discolor in sunlight and in the presence of ferric salts. While salicylic acid is only slightly soluble in water, salicylic acid is known to be solubilized by mixing with various diluents, including alcohol, sodium tetraborate (borax), sodium carbonate or sodium bicarbonate. The reaction with these solubilizing agents both eliminates the crystalline nature of the salicylic acid and buffers the resulting solution to any degree desired, so that a product may be prepared which is close to skin pH. Sodium tetraborate has been preferred in some conventional preparations.

Additionally, in conventional applications preferably, salicylic acid and a solubilizing agent are heated to about 45-50° C. in an aqueous mixture until a clear solution is obtained, with best results achieved by heating with strong agitation. The resulting solutions do not recrystallize, even when subjected to several freeze-thaw cycles (−20 to +45° C.), and no color change occurs during such cycles.

The instant invention provides a surprising and viably economic formulas with ease of preparation with such desirable physical attributes as above mentioned of an acne treatment composition comprising salicylic acid solubilized in a variety of agents, which can be inorganic and organic bases, such as alkali and alkaline earth hydroxides, e.g., sodium hydroxide and potassium hydroxide, and alkali and alkaline earth metal salts of organic acids, such as monosodium citrate, or trisodium citrate, disodium citrate, and dissolved in a cosmetically acceptable aqueous medium to yield desired pH, and prepared at ambient temperature requiring no external heat application. The compositions are easily prepared in batchwise manner for introduction into drug application products.

The term “cosmetically acceptable aqueous medium” as used herein means that of human epidermis compatible drug carrier, and which is as generally known. A drug carrier is any substrate used in the process of drug delivery which serves to improve the selectivity, compliance, effectiveness, and/or safety of drug administration. Drug carriers are primarily used to control the release of a drug into systemic circulation. This can be accomplished either by slow release of the drug over a long period of time (typically diffusion) or by triggered release at the drug's target by some stimulus, such as changes in pH, application of heat, and activation by light. Drug carriers are also used to improve the pharmacokinetic properties, specifically the bioavailability, of many drugs with poor water solubility and/or membrane permeability.

A wide variety of drug carrier systems have been developed and studied, each of which has unique advantages and disadvantages. Some of the more popular types of drug carriers include liposomes, polymeric micelles, microspheres, and nanoparticles. Different methods of attaching the drug to the carrier have been implemented, including adsorption, integration into the bulk structure, encapsulation, and covalent bonding. Different types of drug carrier utilize different methods of attachment, and some carriers can even implement a variety of attachment methods. Any of such known methods, or as yet unknown, are contemplated for use herein.

The composition of the invention can contain about 0.01% to about 35% by weight salicylic acid, preferably from about 0.5 to about 20% by weight, and more preferably about 0.5 to about 5% by weight. For solubilization agent, from about 0.01% to about 65% by weight of one or more solubilizing agents will preferably be used. In order to operate within FDA guidelines for over-the-counter medications, the compositions preferably contain in some formulations from about 0.5 to 2.0% by weight salicylic acid.

Solubilizing agents contemplated herein are preferably selected from alkali or alkaline earth metal hydroxides and/or organic acid salts, or a mixture thereof of two or more of said solubilizing agents, in an amount(s) sufficient to fully or desirably solubilize the salicylic acid (in an absence of alcohol) in a cosmetically aqueous medium in which the salicylic acid and solubilizing agent(s) are dissolved.

In preferred embodiments, the composition of the invention may be formulated, by way of example, into a clear solution, a gel, a cream or a water-in-oil emulsion. When the salicylic acid is to be formulated into a clear solution as described above for direct application or application with a pad, the salicylic acid and solubilizing agent will be present in a cosmetically acceptable medium, generally containing one or more solubility enhancing agents, such as, but not limited to, surfactants and hydrotropes, for example, PPG-5-ceteth-20, ammonium xylene sulfonate or ammonium lauryl sulfate, among others. Ammonium xylene sulfonate and ammonium lauryl sulfate are preferred, and are preferably used in combination. Some non-limiting examples of other commonly used surfactants and emulsifying agents for preparation of gels and creams, include sodium lauryl sulfate, ammonium laureth sulfate, disodium lauryl sulfosuccinate, cocoamphocarboxyglycinate, decyl polyglucoside, cetearyl alcohol, stearyl alcohol, cocamidopropyl betaine, decyl glucoside, glyceryl cocoate, sodium cocoyl isethionate, almond glycerides, sodium lauryl sulphoacetate, sodium lauroyl sarcosinate, sodium methyl cocoyl taurate, sucrose cocoate, polysorbate 20, polysorbate 80, xanthan gum, cellulose, Polyglyeeryl-3 Triolivate, Sorbitan Isostoarate Octyidociecyl Oleate, Glycereth-18 Polyhydroxystearate, Cetyl PEG/PPG-10/1 Dimethicone and Hydroxyethyl Acrylate/Sodium Acryloydimethyl Tauate Copolymer. Other components of the medium may include natural plant botanical extracts, preservatives and fragrances, citric acid and a chelating agent such as tetrasodium EDTA.

As mentioned, the invention also contemplates the use of one or more preservatives commonly employed in cosmetics, and which are known to preserve a formulation, ensure the durability of cosmetic products, and in formulations containing water are efficacious in arresting microorganisms' development during formulation, shipment, storage or consumer use. Preferred for use herein are broad-spectrum preservatives effective at very low concentrations, for example, about 0.001 wt/wt % to about 1.000 wt/wt % of a formula, and which can include, without limitation:
Aldehydes, such as formaldehyde, DMDM hydantoin, imadozolidinyl urea, diazolidinyl urea: safeguard against bacteria and some fungi
Glycol ethers, such as phenoxyethanol and caprylyl glycol: safeguard against some bacteria
Isothiazolinones, such as methylisothiazolinone: safeguard against bacteria and fungi
Organic acids, such as benzoic acid, sorbic acid, levulinic acid, anisic acid: safeguard against fungi and some bacteria
Additional preservatives such as thymol, O-cymen-5-ol and phenylpropanol; antibacterial and antifungal activity as well as preservative boosting ability
   Parabens, such as methylparaben, ethylparaben, propylparaben, are also commonly used as preservatives in cosmetics although there is currently a concern as to whether they may have an effect on human health. Accordingly, glycol ethers, such as phenoxyethanol, are preferred insofar as they are considered a milder alternative to traditional preservatives and a paraben replacer. Such glycol ethers may be combined with, for example, caprylyl glycol, sorbic acid, potassium sorbate, benzoic acid, or EDTA to enhance broad spectrum efficacy. Various antioxidants are also contemplated, such as, without limitation, vitamin E, vitamin C derivaitives, and grapefruit seed extract. While preferred ranges of concentration are recited, any effective amount that is safe for human health is also contemplated for use in the invention.

Multi-active compounds are also contemplated for use herein, such as ethylhexylglycerin, a type of alkyl glyceryl ether, which is used for its surfactant, emollient, skin-conditioning and antimicrobial properties, and preferred for use in amounts of from about 0.001% wt/wt to about 1.000% wt/wt, although any effective amount is contemplated for use.

Preferred fragrances for use herein include without limitation, an olfactory agent in amounts of from about 0.001% wt/wt to about 10.000% wt/wt and/or menthol used in many personal care products as both fragrance and for its analgesic properties, and preferred from use in amounts of from about 0.001 wt/wt % to about 1.000 wt/wt %, and, again, such are contemplated for use herein in any effective amount.

Further contemplated for use herein is simethicone (antifoaming agent), known for use in lubrication and antifoaming application of topical preparations, anti-whitening properties in acne and other dermatological ointments, creams and lotions, and preferred for use in amounts of from about 0.001% wt/wt to about 1.000% wt/wt, although any effective amount is contemplated for use.

In the embodiment of the invention wherein the composition is formulated as a clear solution, the composition may be applied directly to the affected area, or may be applied with a non-woven pad as is well known in the art. In particular, the composition may be dispensed from a container containing one or more non-woven pads which are saturated with the solution.

In the embodiments wherein the composition is formulated as a cream or gel, the composition may be applied directly to the affected area with a designed spot applicator such as, but not limited to, a roll on applicator, a thin brush applicator, a hand held sponge applicator or the like.

The following non-limiting examples are presented to better illustrate some preferred embodiments. It is to be understood that the disclosed embodiments described herein are merely exemplary of the invention, which can be embodied in various forms. Therefore, specific details disclosed herein are not intended to be interpreted as limiting, but merely as a basis for the claims and as a representative basis for teaching one of ordinary skill in the art to variously employ the invention in virtually any appropriately detailed composition as limited by one's own imagination. Various modifications and changes are contemplated without departing from the spirit of the invention and within the scope and range of equivalents of the claims.

### EXAMPLES

### Example 1

A clear solution is prepared with the following composition:

Active Ingredients

| Component | % by weight |
|---|---|
| salicylic acid | 0.001-33.000 |

Inactive Ingredients

| Component | % by weight |
|---|---|
| Purified Water | q.s. to 100% |
| Ammonium Lauryl Sulfate | 0.001-5.000 |
| Ammonium Xylenesulfonate | 0.001-7.000 |
| Tetrasodium EDTA | 0.001-1.000 |
| Preservative | 0.001-1.000 |
| Antifoaming agent | 0.001-1.000 |
| Olfactory Agent | 0.001-10.000 |
| Menthol | 0.001-1.000 |
| Ethylhexylglycerin | 0.001-1.000 |
| Botanical extracts | 0.001-25.000 |
| Sodium citrate | 0.001-15.000 |
| Sodium Hydroxide | 0.001-15.000 |
| Final formula concentration must add to | 100% |

### Example 2

A clear solution is prepared with the following composition:

Active Ingredients

| Component | % by weight |
|---|---|
| salicylic acid | 31.000 |

Inactive Ingredients

| Component | % by weight |
|---|---|
| Purified Water | 48.82500 |
| Ammonium Lauryl Sulfate | 3.2000 |
| Ammonium Xylenesulfonate | 6.0000 |
| Tetrasodium EDTA | 0.0100 |
| Preservative | 0.5000 |
| Antifoaming agent | 0.0100 |
| Olfactory agent | 0.0250 |
| Menthol | 0.0500 |
| Ethylhexylglycerin | 0.001-1.000 |
| Botanical extracts | 0.1000 |
| Sodium citrate | 2.1400 |
| Sodium Hydroxide | 8.1400 |
| Final formula concentration must add to | 100% |

### Example 3

A gel is prepared with the following composition:

Active Ingredients

| Component | % by weight |
|---|---|
| salicylic acid | 0.001-10.000 |

Inactive Ingredients

| Component | % by weight |
|---|---|
| Purified Water | q.s. to 100% |
| Ammonium Lauryl Sulfate | 0.001-5.000 |
| Ammonium Xylenesulfonate | 0.001-7.000 |
| Tetrasodium EDTA | 0.001-1.000 |
| Preservative | 0.001-1.000 |
| Antifoaming agent | 0.001-1.000 |
| Olfactory Agent | 0.001-10.000 |
| Menthol | 0.001-1.000 |
| Glycerin | 0.010-10.000 |
| Xanthan Gum | 0.010-1.5000 |
| Ethylhexylglycerin | 0.001-1.000 |
| Botanical extracts | 0.001-25.000 |
| Sodium citrate | 0.001-15.000 |
| Sodium Hydroxide | 0.001-15.000 |
| Final formula concentration must add to | 100% |

### Example 4

A cream is prepared with the following composition:

Active Ingredients

| Component | % by weight |
|---|---|
| salicylic acid | 0.001-10.000 |

Inactive Ingredients

| Component | % by weight |
|---|---|
| Purified Water | q.s. to 100% |
| Ammonium Lauryl Sulfate | 0.001-5.000 |
| Ammonium Xylenesulfonate | 0.001-7.000 |
| Tetrasodium EDTA | 0.001-1.000 |
| Preservative | 0.001-1.000 |
| Antifoaming agent | 0.001-1.000 |
| Olfactory Agent | 0.001-10.000 |
| Menthol | 0.001-1.000 |
| Glycerin | 0.010-10.000 |
| Xanthan Gum | 0.010-1.5000 |
| Polyglyceryl-3 Triolivate, Sorbitan Isostearate, | 0.010-10.000 |
| Octyldodecyl Oleate | |
| Ethylhexylglycerin | 0.001-1.000 |
| Botanical extracts | 0.001-25.000 |
| Sodium citrate | 0.001-15.000 |
| Sodium Hydroxide | 0.001-15.000 |
| Final formula concentration must add to | 100% |

Procedures for aqueous solutions, cream and gel (Ambient temperature):
1. In main vessel, water and alkaline metals salts are added and mixed until solids are dissolved.
2. Salicylic acid is next added to main vessel. Mixing is performed at room temperature and until completely dissolved.
3. Mix Xanthan gum with glycerin and water, add to main tank when fully hydrated. Xanthan gum is a representative of a preferred structuring agent for use in the invention. One of skill in the art will appreciate that other structuring agents may be used when the formulation calls for a structuring agent.
4. In a separate vessel prepare premix: add the detergents/surfactants, antifoaming agent, chelating agent, flavors, and preservatives. Mix at room temperature. For the cream or gel formulations, mix into the organic phase any desired or required emulsifiers.
4. Transfer premix to main vessel. Rinse side vessel with small amount of water, add the rinse to main vessel and mix as desired.
5. Conduct testing on the finish formula before packaging.
6. Package the formula.

### Example 5

A water in oil emulsion is prepared with the following composition:

Active Ingredients

| Component | % by weight |
|---|---|
| salicylic acid | 0.001-20.000 |

Inactive Ingredients

| Component | % by weight |
|---|---|
| Purified Water | q.s. to 100% |

Phase A

| | |
|---|---|
| Squalane | 0.001-20.000 |
| Dimethicone | 0.001-20.000 |
| Cetyl PEG/PPG-10/1 Dimethicone | 0.010-5.000 |
| Olfactory Agent | 0.001-10.000 |
| Fumed Silica | 0.001-5.000 |
| Phase BTetrasodium EDTA | 0.001-1.000 |
| Preservative | 0.001-1.000 |
| Zinc Chloride | 0.001-1.000 |
| Menthol | 0.001-1.000 |
| Glycerin | 0.010-10.000 |
| Ethylhexylglycerin | 0.001-1.000 |
| Sodium citrate | 0.001-15.000 |
| Sodium chloride | 0.001-1.000. |
| Sodium Hydroxide | 0.001-15.000 |
| Final formula concentration must add to | 100% |

Procedure for Alcohol Free; Water in Oil Emulsion (Ambient Temperature)

| | |
|---|---|
| 1 | Disperse the emulsifier into the oils & fragrance in phase A with mixing |
| 2 | Disperse the silica into Phase A |
| 3 | Prepare Phase B separately, adjust pH to 3.5-5, if necessary, using |
| | sodium citrate or sodium hydroxide |
| 4 | Add Phase B very slowly into Phase A while stirring intensively. |
| 5 | Homogenize to finish the emulsification. |
| 6 | Fill into an appropriate container (tube, jar, pen applicator etc.) |

While the invention has been described with particular reference to some preferred embodiments in the interest of complete definiteness, it is to be understood that it may be embodied in a variety of forms diverse from these specifically shown and described without departing from the spirit and scope of the invention and defined by the appended claims.

What is claimed is:

1. A composition for topical application to human skin comprising by weight:
about 0.01% to about 35% salicylic acid,
about 0.01% to about 65% solubilizing agent selected from one or more alkali and alkaline earth hydroxides, alkali and alkaline earth metal salts of organic acids,
a cosmetically acceptable aqueous medium in which the salicylic acid and solubilizing agent are dissolved,
wherein said solubilizing agent is present in an amount sufficient to solubilize the salicylic acid in the absence of alcohol.

2. The composition of claim 1 wherein the salicylic acid is present in an amount from about 0.5% to about 33% by weight.

3. The composition of claim 1 wherein the cosmetically acceptable aqueous medium comprises at least one surfactant.

4. The composition of claim 3 wherein said at least one surfactant is selected from one or more of PPG-5-ceteth-20, ammonium xylene sulfonate and ammonium lauryl sulfate.

5. The composition of claim 1, wherein the cosmetically acceptable medium contains salts of citric acid.

6. The composition of claim 1, wherein the cosmetically acceptable medium contains a chelating agent.

7. The composition of claim 1 in combination with a non-woven pad as a dispensing medium.

8. The composition of claim 7 wherein said pad is saturated with said composition, and is situated within a container for storage prior to use.

9. The composition of claim 1 which is alcohol free.

10. The composition of claim 1 which is prepared at ambient temperature.

11. The composition of claim 9 which is prepared at ambient temperature.

12. A method for preparing a composition for topical application to human skin comprising, one or more alkali or alkaline earth metal hydroxides and/or organic acid salts with sufficient water for dissolution,
adding a desired amount of salicylic acid and mixing at ambient temperature until dissolved,
and/or optionally adding a solubilizing agent composition and mix at room temperature to dissolve.

13. The method of claim 12 where the solubilizing agent mixture comprises one or more of a surfactant, antifoaming agent, chelating agent, olfactory agent and or a preservative.

14. The method of claim 13 wherein the salicylic acid is present in an amount from about 0.01% by weight to about 35% by weight.

15. The method of claim 14 wherein the salicylic acid is present in amount of from about 0.5% by weight to about 2% by weight.

16. A method of treatment of a human with a skin condition comprising applying the composition of claim 1 to skin of the human, wherein the salicylic acid is present in the composition in an amount effect to reduce a symptom of the skin condition.

17. The composition of claim 1, wherein the solubilizing agent is present in the composition in an amount sufficient to solubilize the salicylic acid when the salicylic acid is mixed with a solution comprising the solubilizing agent and water at a room temperature in the range of about 18-24° C.

18. The composition of claim 1, wherein the solubilizing agent comprises an alkali or alkaline earth metal hydroxide.

19. The composition of claim 1, wherein the solubilizing agent comprises an alkali or alkaline earth metal salt of an organic acid, and wherein the organic acid is selected from the group consisting of citric acid, glycolic acid, lactic acid, Tartaric acid, and malic acid.

20. The method of claim 10, wherein the composition is prepared at a room temperature in the range of 18-24° C. without heating.
